(19) United States
(12) Patent Application Publication
(10) Pub. No.: US 2010/0280428 A1
(43) Pub. Date: Nov. 4, 2010

(54) MEDICAL DEVICE FOR SHRINKING OPEN WOUNDS

(76) Inventors: Alan D. Widgerow, Irvine, CA (US); Malan de Villiers, Irene (ZA)

Correspondence Address: FISH & ASSOCIATES, PC;ROBERT D. FISH, 2603 Main Street, Suite 1000, Irvine, CA, 92614-6232, US

(21) Appl. No.: 12/767,743

(22) Filed: Apr. 26, 2010

Related U.S. Application Data
(60) Provisional application No. 61/172,718, filed on Apr. 25, 2009.

(51) Int. Cl. A61F 13/02

(52) U.S. Cl. 602/53

(57) ABSTRACT

A wound closure device has internal and external components designed to close, shrink or approximate an open wound. The internal component preferably comprises sachets filled with highly absorbent material such as carboxymethylcellulose, disposed at the surface of the wound bed to absorb exudate from the wound. The external components preferable include paddles that adhere to the skin, and form part of, or have a mechanism to couple to an elastic tensioning fabric. The internal component is placed between the open wound and the elastic tensioning fabric, such that expansion of the internal component applies pressure on the wound base. It is contemplated that the two components will work synergistically together to create ‘micro-deformation’ forces that stimulate granulation tissue.

### RELATED APPLICATIONS

The present application is a continuation application of U.S. provisional patent application, Ser. No. EFS ID 5219601; Application No. 61/172,718, filed Apr. 25, 2009, for MECHANICALLY ASSISTED TISSUE CONTRACTION AND HEALING (MATCH) DEVICE, incorporated by reference herein, and for which benefit of the priority date is hereby claimed.

### FIELD OF THE INVENTION

The application relates to a medical device used to approximate an open wound, more particularly shrinking the open wound in multiple dimensions.

### BACKGROUND OF THE INVENTION

An open wound refers to one that has not, or cannot be, closed primarily by simple suturing or binding together of the wound edges by staples, tapes or glues. It varies between small wounds that do not close over a long period (usually 4 weeks) referred to as chronic wounds or larger wounds that can not close due to the nature of injury—war wounds, major trauma, surgical complications where wounds have burst open. The background to many of the wounds be they large or small, can relate to diseases such as diabetes, circulatory problems, pressure sore and the like. On the other hand there can be no background disease process but the wound is just unable to be closed primarily.

Traditionally these open wounds are treated with various dressings that would aim at cleaning the wound base, preventing infection and promoting healing. The wound is thus prepared for further surgical closure (skin graft/transplantation or skin flap closure) at a later date once it is clean or the patient is stable enough to undergo surgery, or the dressings are continued for a protracted period of time until the wound eventually closes on its own (secondary intention). This technique does nothing to aid the wound in closing—it merely prepares the wound bed for further surgery or keeps the wound clean while healing takes place over an extended period of time. The latter technique is problematic as during the long period of closure, the wound is subject to recurrent infections and protracted dressings are applied at great inconvenience and expense.

An alternative dressing/device that is being used currently for difficult to close/heal wounds is that involving Negative Pressure Wound Therapy (NPWT). This involves using a sponge or gauze dressing on the base of the wound, attaching a pipe to the dressing, sealing the wound and dressing and attaching the opposite end of the pipe to a machine that applies suction to the wound. This machine is electrically or battery operated and can vary in size and complexity. This suction can be intermittent or continuous. The suction applied to the wound is commonly referred to as suction, where ideal negative pressures are described as −50 to −150 mmHg relative to ambient pressure. These negative pressures are thought to ‘suck’ on the wound bed surface creating what is called ‘micro-deformation’ of the wound bed surface, which is touted to encourage new healing tissue (granulation tissue). An added advantage of the technique is the sucking up of the secretions (exudate) from the wound. This exudate can be detrimental to wound healing in excessive amounts. Manufacturers also claim advantages of wound contraction (approximation of the wound edges) of approximately 10% in a week. Thus, the wound device keeps the wound from over-saturating with secretions, encourages new tissue growth and causes a certain amount of contraction. The disadvantage is the device is costly, is electrically operated, is cumbersome even in its portable form, and brings about very limited wound contraction or shrinkage, so that further surgery is often still necessary to heal the wounds in most cases.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

Other examples of prior art that should be mentioned at this stage are devices designed to close a wound that has been left open. These are divided into non-invasive devices (those that do not penetrate the skin) and invasive devices (those that use needles, hooks, sutures or the like to penetrate the skin as traction to close the skin).

Non-invasive wound closure devices are primarily designed to close wounds that close together where little force is involved in approximating the wound edges—thus variations of tapes, glues, zip dressings and the like that have been designed to obviate the need for suturing a wound, that approximate the wound edges and hold them together. This would not be applicable or effective to the type of wound described above due to the greater complexity, increased size of the wound opening and the force that would be needed to close such a wound with the non-invasive device described.

Invasive devices have been designed to close or approximate larger wounds. These involve variations from needles that are inserted into the wound edges and then used as traction with sutures, ties, wires, tapes, to hooks that are inserted into the skin surface as anchors for suture or thread traction or similar such designs. These invasive devices necessitate the use of local or general anesthesia for their application; they are painful for the patient and leave extremely unsightly scars on the skin when the process is over. These are major disadvantages, and have resulted in very infrequent use of this modality.

Current methods of closing complex open wounds are either invasive and expensive and require anesthesia (surgery, needles inserted in the skin and traction applied); or impractical for large wounds (tape closures, small tension bands); or have minimal impact on closing the wound (NPWT approximates the edges about 10% in a week) but are useful for preparing the wound bed for surgery. There is no current device that approximates the wound edges effectively while promoting wound bed preparation by stimulating granulation tissue, that is non-invasive (can be done while the patient is in bed or mobile); requires no anesthesia, is extremely cost-effective and results in shrinkage of the wound in all dimensions often attaining full closure. Hirshowitz et al. (Israel Patent No. 097225) developed a skin stretching device that consists of two “U” shaped arms with sharp cutting hooks that engage two long pins that are threaded through the dermis on either side of the wound. The arms can then be pulled closer to each other and stretch the skin by a screw turned by a tension knob. Bashir (British Journal of Plastic Surgery 1987,40,582-587-Wound Closure By Skin Traction) applied the mechanical creep effect by threading steel wires through the edges of the defect and then over a period of days twisted them to apply tension to the skin. Cohen et al. discloses a Suture Tension Adjustment Reel that is applied to gradually tighten a suture that is passed through the two opposing skin edges of the wound (Dermatol Surg Oncol 1992;18:112-123 and U.S. Pat. No. 5,127,412). An apparatus for closing wide skin defects can be used with two long interdermal needles configured for insertion underneath skin close to margins of a skin defect is described in U.S. Pat. No. 5,893,879. Some methods have been developed for sternum repair based on using a band assembly (with a needle) secured by a buckle mechanism in a closed loop configuration about the sternum portions. Typical assemblies are described in U.S. Pat. Nos. 5,462,542, 5,330,489, 5,356,412 and more. The devices described by the above mentioned patents could not be used effectively for large wound closure. All these techniques involve invasive use of hooks or needles inserted into the skin with problems of anesthesia, scarring, pain and limited efficacy.

Other prior art includes U.S. Pat. No. 4,815,468 March 1989 to Annand, U.S. Pat. No. 4,825,866 can 1989 to Pierce; U.S. Pat. No. 5,665,108 September 1997 to Galindo, U.S. Pat. No. 70,014,837 August 2006 to Johnson; U.S. 6,471,715 October 2002 to Weiss, U.S. Pat. No. 6,726,706 April 2004 to Dominguez, U.S. Pat. No. 6,831,205 December 2004 to Lebner, U.S. Pat. No. 6,120,525 December 2000 to Westcott, and U.S. Pat. No. 4,535,772 August 1985 to Sheehan. These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

It is contemplated herein that an ideal device for the complex open wound described above would be one that produces the results of the NPWT, but with greater wound contraction and use of a non-invasive, non-electronic, more cost-effective, more comfortable device. Thus, it would deal effectively with the wound bed as well as encouraging wound edge approximation—that is, total wound shrinkage. Among other things;

It would be advantageous to provide efficient absorption of wound exudate;

It would also be advantageous to provide stimulation of granulation tissue;

It would further be advantageous to provide significant closure of the wound edges;

It would further be advantageous to provide positive changes in blood flow at the base, surrounding tissue and edges of the wound;

It would further be advantageous if the combination of internal and external devices result in perfusion changes and stimulation of granulation that promote shrinkage and closure of the entire wound;

It would further be advantageous if this positive pressure caused by the combination of internal and external components of the device on the wound bed promotes cell division and new granulation tissue formation; and

Finally it would be advantageous to provide overall three dimensional wound shrinkage or complete closure not accomplished by any other device on the market today.

### SUMMARY OF THE INVENTION

The inventive subject matter provides apparatus, systems and methods in which a medical device for shrinking and approximating open wounds comprises an external component with at least first and second adherent paddles, an elastic material that spans the distance between the paddles, and a wound exudate absorbing material, disposed to transmit pressure to a surface of the wound.

In one aspect there is provided a non-invasive mechanical device that has internal and external components. There is provided in the internal component sachets containing absorbent material such as CMC that fit the surface of the wound bed and effectively absorb the exudate. The external device consists of paddles placed (adherent to skin) on either side of the wound with a tension fabric connecting the two paddles resulting in wound approximation. The two components work synergistically together—as the sachets expand they impinge on the external approximating device which results in downward pressure on the wound creating ‘micro-deformation’ forces that stimulate granulation tissue. Perfusion also improves adding to the positive wound healing effect and creating overall effective wound shrinkage.

Preferred devices are designed to shrink the entire wound area by promoting healing of the wound from its base, encouraging new healthy granulation tissue and by approximating the open edges of the wound diminishing the open exposed area of the wound significantly. The device is applied to a wound which is non-healing, or electively left open for medical or emergency (transport) reasons or mechanically unable to be closed.

The external portion of preferred embodiments comprise the following:

One element (paddle) of compliant material comprising an adherent posterior surface which sticks to the skin surface on either side of the open wound; the anterior surface comprises hook like structures on which the second element abuts and fixes while under traction.

A second element comprising strong elasticized or tension stretch fabric, or rubber or silicone, The element can have holes uniformly situated in the material or loop like structures that will adhere to the hooks or fabric (with loops) that is embedded within the silicone type structure that sticks to the element described in a.); The holes or fabric being intended to come into abutment on the hook like structures of element one. This second element serves as a connecting or bridging piece between the two elements that are adherent to the skin on either side of the wound. The element two is advanced as required all the time increasing traction on the wound edges and causing approximation of the wound edges. The element two is comprised of elasticized fabric which further encourages approximation of the skin edges on either side of the wound. This device allows a non-invasive (no needles, staples, sutures etc), gradual approximation of the wound edges.

The internal portion of preferred embodiments comprise a carboxymethylcellulose (CMC) or equivalent absorptive material dressing made up in various forms from a flat interface dressing to separate sachets filled with CMC. This is placed on the surface of the wound primarily to deal with the exudate (fluid that is produced by the wound) that can delay healing if excessive. CMC can absorb up to 200 times its weight and is thus very effective for wound exudate. Sachets have been especially designed for the system in different sizes to be used in different sized wounds. They are similar in appearance to a tea bag filled with CMC instead of tea. The material of the sachets vary from nylon to paper and can include fabrics of multiple consistencies and varying textures depending on the wound bed interface reaction that is desired. The wound is packed with this dressing; the external approximator appliance is applied over the dressing. Once the CMC begins absorbing the fluid it swells considerably and the resultant force from the surface approximating device on the swollen CMC sachets is directly applied to the wound surface. Unexpectedly, our results have shown us that this positive pressure exerted on the base of the wound also creates ‘micro-deformation’ of the tissue at the base of the wound thus stimulating new healing (granulation) tissue. This can be assessed by clinical inspection of the granulation tissue on the wound bed surface which can be obvious to the naked eye (FIG. 3), or by histological assessment.

FIG. 1 shows an area of skin 100 having granulation tissue growth 110 on surface of wound bed 120 (bright red areas of fresh tissue growth).

The combination of the above modalities results in the following positive outcomes: 1. Efficient absorption of wound exudate; 2. Stimulation of granulation tissue; 3. Significant closure of the wound edges; 4. Unexpected positive changes in blood flow at the base, surrounding tissue and edges of the wound (elaborated below); and 5. Overall three dimensional wound shrinkage not accomplished by any other device on the market today.

The combination of these modalities also provides for a mechanical alternative to the negative pressure wound therapy (NPWT) devices (electric) that are used today. It can also be used with NPWT. It is effective in healing and cost effective to use. It has major advantages of significantly more external wound closure than NPWT.

Some of the unexpected elements of the inventive subject matter relate to the pressure effects and the perfusion effects when this combination is used. The external device, by its mechanical pressure on the skin and the wound edges, would be expected to decrease wound perfusion in the wound base and edges which would traditionally be considered to be problematic. Perfusion studies show that perfusion is temporarily decreased at the wound edges and base, but the surrounding increase in perfusion soon overrides this local effect and has no negative effect on healing, but in fact encourages healing by creating a temporary oxygen gradient (decreased oxygen at the base of the wound) and stimulates increased perfusion secondarily. This perfusion was assessed by radioisotope studies in 7 patients in multiple areas of the wound; typically the perfusion was seen to increase close to threefold from day 2 to 3 after application of the device; it then settles to a level 150% above baseline for the following few days (Graph 1)(V Chetty, University of the Witwatersrand Feb.-Oct. 2009). See FIG. 2:

Thus the pressure on the base of the wound is positive pressure, and not negative pressure as has been described for other devices. We have discovered that positive pressure on the wound is at as least as effective as negative pressure, challenging an accepted dogma in current wound therapy.

The novelty of using a combination of internal and external devices for wound shrinkage is apparent. The wound is stimulated to heal from within and simultaneously the external wound edges are significantly approximated. In objective terms, the NPWT is expected to create external wound contraction of 10-15% in one week; the device described in this invention creates external shrinkage of 75% in most cases (FIGS. 3 and 4).

FIG. 3 shows an area of skin 300 with an open abdomen 310.

FIG. 4 shows skin 300 having a dramatic narrowing of the open abdomen 310 of FIG. 3 following application of a preferred device 410.

In addition to that described above, various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

A complete understanding of the present invention can be obtained by reference to the accompanying drawings, when considered in conjunction with the subsequent, detailed description, in which:

FIG. 1 a depiction of an area of skin having granulation tissue growth on surface of a wound bed.

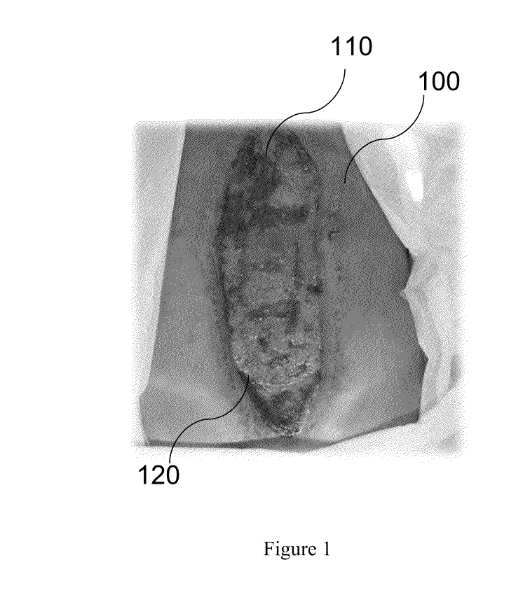

FIG. 2 is graph showing percent perfusion as a function of time.

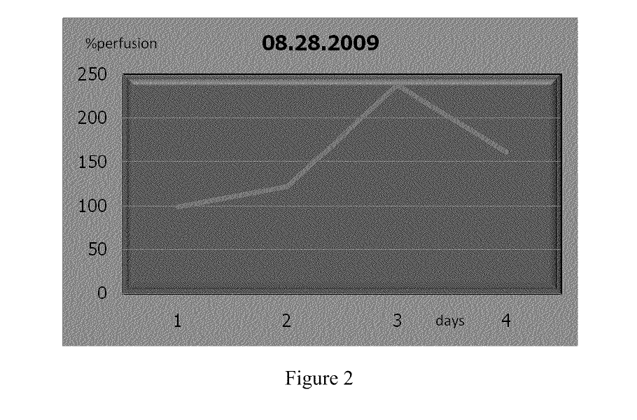

FIG. 3 is a depiction of an area of skin with an open abdomen.

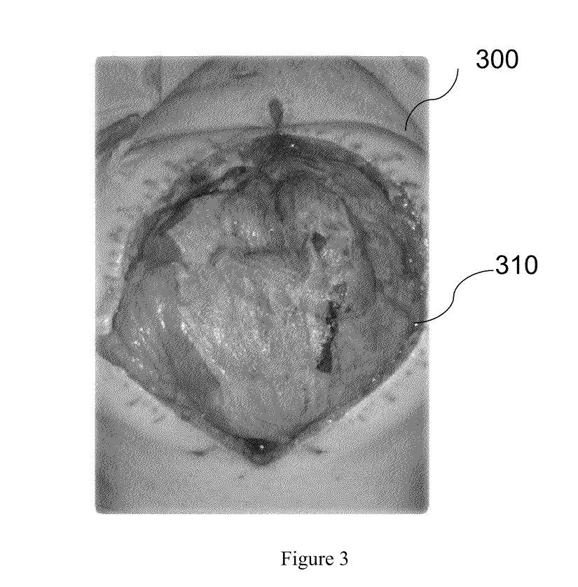

FIG. 4 is a depiction of the area of skin of FIG. 3, following application of a preferred device.

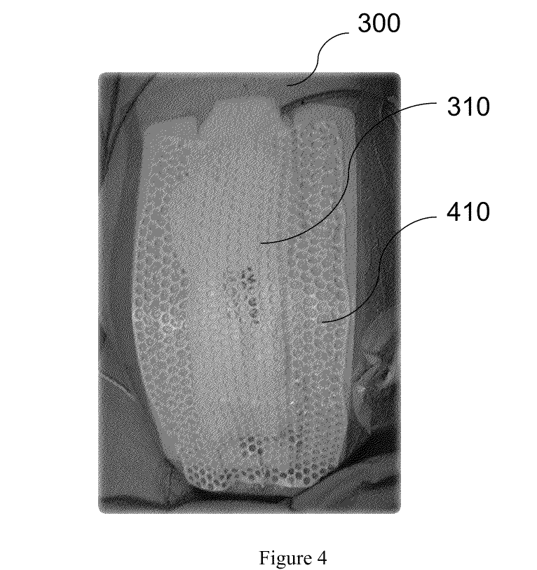

FIG. 5 is a perspective segmented view of the entire embodiment of my invention. On the top is the elasticized material 501 that bridges the gap between the 2 paddles 502 that are attached to the skin surface adjacent to the wound. The posterior surface of the paddles has adhesive to stick to the skin while the anterior surface has hook like structures for attachment of the elasticized silicone impregnated material. In the depth of the wound on the wound bed surface is the sachet filled with absorbent material 503;

FIG. 6 is a cross section of the entire embodiment in place. The first diagram is a perspective of a wound with the sachets with absorbent material placed on the wound bed 603. The paddles 602 are then attached to the skin 604 and the elasticized material 501 attached to the paddles 602. The material is elasticized, can be fabric, silicone, rubber or any grade of elasticized tension producing material. It can have holes or a silicone structure impregnated with fabric that will adhere to the paddles.

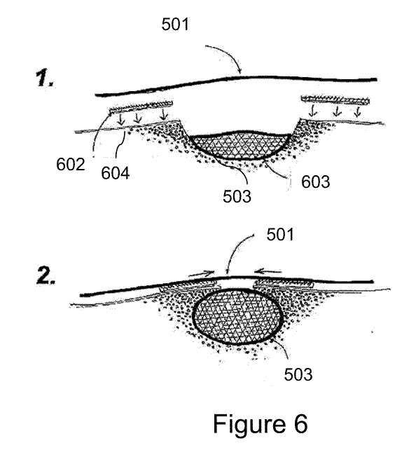

The second diagram of FIG. 6 is a perspective of the sequence of events that occurs when the device is in place: the sachet absorbs wound fluid and increases in size (5). The elasticized bridging material approximates the wound edges and the increased size of the sachets together with the external tightness of the bridging material create pressure on the wound bed surface promoting new tissue formation (granulation tissue) and increased perfusion of the surrounding tissue. The increased pressure on the surrounding skin also appears to move fluid from the skin edges (edema) encouraging movement and approximation of these edges. The combined effects of the device result in three dimensional wound shrinkage and encouraged healing.

### DETAILED DESCRIPTION

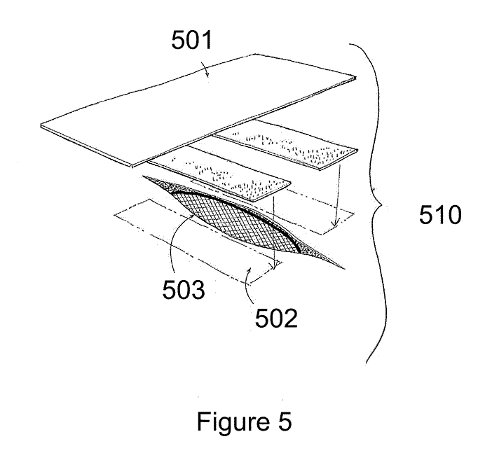

In FIG. 5 the external device 510 generally comprises an elastic covering 501, a first and second skin coupling components 502, and an expandable dressing 503. In especially preferred embodiments the device is entirely non-invasive—no needles need be inserted into the skin, and no sutures need be applied to the skin for use with the device.

The elastic covering 501 preferably comprises an elasticized fabric with or without punched holes, these being intended to fix onto or adhere to the skin hooks on the first element and sequentially advance into new positions over time promoting traction approximation of the wound edges.

Each of the skin coupling components 502 preferably comprises an adherent posterior surface intended to adhere to the skin on either side of the open edge of the wound, and anterior surface comprising ‘hook-like’ protrusions, which when connected to the elastic covering 501 promotes traction approximation of the open wound. The posterior surface of each skin coupling component 502 advantageously presents a sufficient surface to exert a widespread fraction on the skin adjacent to the wound. Each component is placed at an appropriate distance from the wound edge so as to allow traction on the adjacent surface area of unaffected skin without the use of invasive devices such as needles, sutures, staples and the like.

Expandable dressing 503 is selected to enhance healing tissue and absorb exudate, and can include CMC or any other suitable absorbent material, in sufficient quantity to transmit an effective amount of pressure from the elastic covering 501 to the wound surface. This results in positive pressure on the wound bed with demonstrated promotion of healing and increase in perfusion. Numerous variations of the dressing making up the internal component are possible, including the following:
a. The absorbent dressing can include foams formed of a polymeric material, such as polyurethane or polyester as well as PVA open cell polymer material, or other similar material having a pore size sufficient to facilitate wound healing;
b. The absorbent dressing can consist of a molecular sieve drying agent or a hydrogel drying agent. A molecular sieve drying agent can consist of any absorbent granules, powders or beads, for example, Sodium Polyacrylate;
c. The dressing can consist of a hydrogel forming agent upon coming in contact with fluid;
d. The dressing can consist of a Valine type fabric, a flint free microfilament consisting of Polyester and Polyamide and any other suitable soft material;
e. The dressing covering can consist of a polyurethane layer or any non-adherent agent for patient comfort;
f. The paddles and fabric can consist of Velcro hook and loop type fabric with adhesive paddles being made of firm outpouchings such as those seen with Velcro material; and
g. The tension band material that spans the paddles can be made of rubberized material or silicone material. Embedded within this rubber or silicone a fabric of nylon, lycra, cotton or other material can be present allowing it to stick to the paddles.

### Operation

The device is designed for use in an open wound that cannot be closed primarily. It can be used to decrease or “shrink” the size of the open wound in preparation for surgery and final closure, or it can bring about full closure of the wound without further surgery. In both cases, the wound bed is prepared and optimized by absorption of excess secretions, stimulation of healing granulation tissue and encouragement of approximation of the wound edges.

In FIG. 6, the skin coupling components 602 are paddles, which are stuck to the skin on, and aligned at, right angles to the wound edge in parallel with the wound edges approximately 1 inch from the wound edge. The expandable dressing 503 containing highly absorbent material such as CMC are then placed on the wound bed surface, or a few expandable dressings can be used depending on the size of the wound. It is envisaged that different sized expandable dressings will be available. The expandable dressings can then be covered with a simple dry gauze dressing (not shown).

The paddles 602 are then connected with the elastic covering 501. This is done by mating material on the underside of the elastic covering 501 with material on the upper side of the paddles, using hook and loop or other suitable technology. This is done first on one side of the wound, and then on the other side, to provide an appropriate tension that approximates the edges (or at least draws them closer together) without overly obstructing the circulation or displacing the paddles. Appropriate tension can be assessed by observing the skin surface circulation and the status of the netting and paddles. Buckling or excessive tension is to be avoided.

Once absorption of the secretions from the wound bed begins, the expandable dressings begin to swell and exert pressure on the wound bed surface. This encourages micro-deformation of the cells at the wound bed dressing interface encouraging further healing granulation tissue to form. It is envisaged that the dressing will be changed at between 2 and 5 days, but this could be sooner if the wound has a heavy exudate with excessive secretions.

Changing the dressing involves unhooking of the elasticized material from the paddles, removal of the expandable dressings, cleaning of the wound and replacement with new expandable dressings. The sequence as described above is repeated but the skin should now be more closely approximated and replacement of the tension band/netting/elasticized material should further approximate the wound edges. This sequence continues until the wound edges are completely approximated, or until it is elected to continue the treatment with a definitive surgical procedure. This procedure can take the form a simple suturing if the wound edges are close enough, or skin transplantation or another form of skin flap closure. In all cases, it is envisaged that the invention will have prepared and optimized the wound bed while approximating the wound edges too—that is overall shrinkage of the wound size. This is achieved by a new concept of positive wound pressure rather than commonly accepted negative wound pressure together with successful wound edge approximation.

The present inventors contemplate numerous alternatives. For example, the skin coupling components 502 can be of different sizes and configurations to match different size wound defects/openings, and it is contemplated that different sizes or configurations could even be used on a single wound. The skin coupling components 502 can be rectangular, square, crescent shaped or a multitude of other shapes to adapt to unusual wound shapes. The skin coupling components 502 could also have relieving slits cut into their structure to allow a certain amount of bending and flexibility.

Also, the material from which the skin coupling components 502 are made can vary from a soft semisolid material that can adapt to contours to a more rigid material required for increased tension in large defects. They can be manufactured in the form of rolls of material, with skin coupling components 502 cut off the roll according the size needed, or they can be manufactured in a variety of individual sizes. The skin coupling components 502 can be approximated by a series of ‘ball and chain’ threads that wind through the paddles and are tightened in a ratchet type of device.

As another example, the skin coupling components 502 could have different types of connectors to the elastic covering 501 other than Velcro™ type connectors. One could use a single or double row of hooks, or even an appropriate adhesive.

It is also envisaged that materials can improve to such an extent that skin coupling components 502 can be integrated into the elastic covering 501. That is, an adherent elasticized or tension band like material can be placed from one side of the wound to the other with a large overlap over normal skin and once adhered to both sides of the wound, can approximate the edges without separate paddles or other skin coupling components.

Elastic covering 501 can be manufactured from a variety of materials, the main pre-requisites being the ability to withstand tension, and an inherent elasticity or recoil that would encourage approximation of edges of the wound. The covering can be transparent so as to enable an observer to see the dressing beneath it; however variations in colors, texture and pliability are also envisaged.

Although expandable dressing 503 would typically have CMC as its filler material, various other agents can also be used and combinations are possible. Thus, honey, hydrolyzed collagen, foams and gelatinous materials can be used as fillers to the expandable dressings. The expandable dressing material encasing these fillers can be made from nylon, silk, and all variations of microporous materials that exist to date. Thus, expandable dressing 3 could be a “sachet”, but it could also be as simple as a semi-solid sheet manufactured with highly absorbent material that swells with absorption. The expandable dressing can include foams formed of a polymeric material, such as polyurethane or polyester as well as PVA open cell polymer material, or other similar material having a pore size sufficient to facilitate wound healing. The expandable dressing could additionally include a molecular sieve drying agent or a hydrogel drying agent. Such a drying agent could include absorbent granules, powders or beads, for example, Sodium Polyacrylate. Contemplated expandable dressings can also include hydrogel forming agent upon coming in contact with fluid. Still further, contemplated expandable dressings can include a Valine type fabric, a flint free microfilament, such as Polyester and Polyamide, and any other suitable soft material. The expandable dressing covering can consist of a polyurethane layer or any non-adherent agent for patient comfort.

Accordingly the reader will see that the mechanically assisted tissue closure and shrinkage device and its variations of embodiments provide a new technique:
a. That can shrinks the wound in its entirety;
b. That relies on a new concept of positive pressure wound therapy;
c. That involves a non-invasive technique which encourages patient comfort, safety (no anesthesia) and the possibility of ambulatory, non-hospital stay, therapy;
d. That absorbs secretions efficiently and stimulates new healing granulation tissue;
e. That approximates the wound edges much more effectively than other devices on the market (especially those designed for negative pressure wound therapy—NPWT);
f. That is considerably cheaper, as efficient (more efficient in edge approximation) and far less cumbersome than devices designed for NPWT;
g. That introduces a new unexpected effect of positive pressure on granulation tissue, perfusion and overall wound bed preparation, that traditionally was thought to be the exclusive domain of negative pressure wound therapy; and
h. That deals with all aspects of the wound, internal and external to ensure total wound shrinkage in a synergistic manner not previously described.

This synergy provide wound secretion absorption; during the process of absorption the design of the internal segment produces positive pressure on the wound bed; this positive pressure has resulted in stimulation of granulation tissue and improvement of perfusion (blood supply) to the healing area; simultaneously wound edge approximation is extremely efficiently produced by the external segment of the embodiment.

It is contemplated that by use of the inventive concepts here, surgery can be avoided or simplified; the metabolic demands on the patient are reduced due to the shrinkage of the wound; costs of therapy are reduced; patient comfort is increased; simple application obviates the need for specialized staff using the device.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps can be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A medical device for shrinking and approximating an open wound, comprising:
an elastic covering;
a first and second skin coupling components; and
an expandable dressing, disposed such that absorption of wound exudate causes expansion of the dressing, and thereby positive pressure being applied against the wound.

2. The device of claim 1 wherein the elastic covering mates with the skin coupling components using a hook and loop technology.

3. The device of claim 1 wherein the expandable dressing comprises a sachet.

4. The device of claim 3 wherein the sachet contains at least one of carboxymethylcellulose, a molecular sieve drying agent, and a hydrogel forming agent.

5. The device of claim 3 wherein the sachet has a covering comprising a polyurethane, a PVA open cell polymer material, a Valine type fabric, and a flint free microfilament

6. The device of claim 1, where the first and second skin coupling components are incorporated into the elastic covering.
